(19) United States
(12) Patent Application Publication
(10) Pub. No.: US 2020/0377955 A1
(43) Pub. Date: Dec. 3, 2020

(54) USE OF CELL-FREE CIRCULATING RNA (cfRNA) EXPRESSION OF PD-L1 AND ERCC1 IN PLASMA TO MONITOR RESPONSE TO THERAPY IN NSCLC

(71) Applicant: Liquid Genomics, Inc., Culver City, CA (US)

(72) Inventors: Kathleen DANENBERG, Culver City, CA (US); Joshua USHER, Culver City, CA (US); Shahrooz RABIZADEH, Culver City, CA (US)

(21) Appl. No.: 16/624,758

(22) PCT Filed: Jun. 19, 2018
(86) PCT No.: PCT/US18/38198

§ 371 Date: Dec. 19, 2019

Related U.S. Application Data
(60) Provisional application No. 62/522,615, filed on Jun. 20, 2017.
(60) Provisional application No. 62/570,199, filed on Oct. 10, 2017.

(51) Int. Cl. C12Q 1/6886

(52) U.S. Cl. CPC C12Q 1/6886; C12Q 2600/112; C12Q 2600/158; C12Q 2600/106

(57) ABSTRACT

Quantitative levels of ERCC1 cfRNA are used to monitor/predict a clinical response with respect to a disease state of a cancer in a patient subject to treatment with a platinum-based drug. Most typically, the cancer is a NSCLC and the patient is treated with a platinum-based drug. Where treatment also includes immune checkpoint inhibitors, PD-L1 cfRNA may be quantified to further predict treatment outcome.

This application claims priority to copending U.S. provisional patent application Ser. No. 62/522,615, filed Jun. 20, 2017, and claims priority to copending U.S. provisional patent application Ser. No. 62/570,199, filed Oct. 10, 2017.

### FIELD OF THE INVENTION

The field of the invention is compositions and methods of predicting and monitoring treatment response to cancer therapy, especially as it relates to use of cfRNA for PD-L1 and ERCC1 for analysis of treatment response in non-small cell lung cancer (NSCLC).

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Over the last decades, efforts in improving cancer treatment have largely focused on screening, development of new anti-cancer agents, multi-drug combinations, and advances in radiation therapy. In a more recent approach, individual variability of tumors are taken into account to design personalized treatment strategies. One important goal of precision medicine is to identify molecular markers, and especially nucleic acid markers, indicative of therapy selection by analyzing the factors involved in the therapeutic effects and prognosis.

While it was known that nucleic acid molecules from tumor and non-tumor cells can be obtained from blood (see e.g., Clin Canc. Res. (1999) Vol 5, 1961-1965; Canc Res. (1977) 37:646-650), it was not clear whether or not these nucleic acids were associated or bound with any carrier or other structure. Indeed, more recently it was discovered that RNA can originate from various sources, including circulating tumor cells (see e.g., WO 2017/180499), exosomes (see e.g., WO 2015/082372), and carrier proteins (see e.g., WO 2010/079118, or Proc. Nat. Acad. Sci. (1985) 82, 3455). Unfortunately, and possibly due to the different locations/associations of RNA with various carriers or other subcellular structures, accurate quantification of circulating nucleic acids has often been problematic. For example, disease status detection in neuroblastoma using cell free RNA was shown not to be a reliable alternative to whole cell RNA analysis (see e.g., Pediatr Blood Cancer. 2010 Jul. 1; 54(7):897-903).

WO 2016/077709 teaches measurement of various cfRNA and cfDNA species. While being able to detect relatively small quantities of cfRNA from mutated or improperly fused genes in the blood regardless of their particular association, the detected quantities of such RNAs varied significantly. Moreover, it also remained unknown whether any of the detected quantities was a reflection of physiological reality within a cell or a function of stability of the particular RNA in question. For example, data in the '709 publication indicate that the quantities of cfRNA encoding PD-1/PD-L1 is often highly variable and may depend on the sample, patient condition, and other factors. WO 2016/077709 further teaches measurement of various cfRNA and cfDNA species from NSCLC patients at various times during therapy. Notably, and without any stratification, ERCC1 expression was detected in 100% (10/10) of NSCLC patients and 67% (6/9) of the control group, with no significant difference observed in the relative expression of those detected. Indeed, the inventors of the '709 application concluded that ERCC1 expression would exemplify a gene that exhibited no significant difference in expression level across cancer patients and healthy individuals. Consequently, while various methods are known to detect and quantify cfRNA, use of such methods to monitor or predict treatment outcome of a cancer with respect to a disease state (e.g., progressive disease (PD), stable disease (SD), partial response (PR)) with a specific drug have been elusive.

Therefore, even though numerous methods of nucleic acid analysis from biological fluids are known in the art, all or almost all of them suffer from various disadvantages. Thus, there still remains a need for improved systems and methods to monitor or predict treatment outcome of a cancer with respect to a disease state using cfRNA.

### SUMMARY OF THE INVENTION

The inventive subject matter is directed to compositions and methods of quantitating expression of ERCC1 cfRNA in a bodily fluid of a patient diagnosed with cancer. Notably, the inventors discovered that ERCC1 cfRNA quantities can be used to monitor or predict a clinical response with respect to a disease state of a cancer in a patient subject to treatment with a platinum-based drug.

In one aspect of the inventive subject matter, the inventors contemplate a method of monitoring or predicting clinical response with respect to a disease state of a cancer in a patient subject to treatment with a platinum-based drug. Most typically, such method will include a step of quantitating relative expression of ERCC1 cfRNA in a bodily fluid of the patient diagnosed with the cancer (e.g., lung cancer such as NSCLC).

In some embodiments, the relative expression of ERCC1 cfRNA is quantified relative to beta-actin RNA or Universal Human Reference RNA. Where desired, an indication may be generated when the relative expression of ERCC1 cfRNA is above a level indicative for stable disease (e.g., indication is predicted resistance or lack of response to the treatment with the platinum-based drug). Alternatively, or additionally, an indication may be generated when the relative expression of ERCC1 cfRNA is at or below a level indicative for stable disease. In such case, the indication may be predicted partial or full response to the treatment with the platinum-based drug.

Contemplated methods may also include a step of quantitating relative expression of PD-L1 cfRNA in the bodily fluid of the patient diagnosed with the cancer, for example, wherein the patient is further subject to treatment with a checkpoint inhibitor. Moreover, it is contemplated that the methods presented herein may also comprise, at least one week after the step of quantitating relative expression of ERCC1 cfRNA, a second step of quantitating the relative expression of ERCC1 cfRNA in the bodily fluid. Therefore, where desired, a dynamic change of ERCC1 cfRNA may be detected using the steps of quantitating the relative expression of ERCC1 cfRNA in the bodily fluid.

In further contemplated embodiments, the disease state is partial response when the relative expression is at or below 1.5, the disease state is stable disease when the relative expression is between 1.8 and 2.8, and/or the disease state is progressive disease when the relative expression is at or above 3.8.

Therefore, and viewed from a different perspective, the inventors also contemplate use of ERCC1 cfRNA to monitor or predict a clinical response with respect to a disease state of a cancer in a patient subject to treatment with a platinum-based drug, wherein the ERCC1 cfRNA is quantitatively measured ex vivo in a bodily fluid sample of the patient.

For example, where the cancer is a lung cancer, the platinum-based drug may be carboplatin. Most typically, ERCC1 cfRNA is quantitatively measured as relative expression against expression of at least one reference gene (e.g., beta actin or Universal Human Reference RNA). In addition contemplated uses may further include a step of quantitatively measuring PD-L1 cfRNA ex vivo in the bodily fluid sample of the patient, especially where the patient is also subject to treatment with a checkpoint inhibitor.

Various objects, features, aspects and advantages of the inventive subject matter will become more apparent from the following detailed description of preferred embodiments, along with the accompanying drawing.

### BRIEF DESCRIPTION OF THE DRAWING

FIG. 1 is an exemplary graph depicting relative ERCC1 expression in lung cancer patients with NSCLC in response to platinum-based therapy.

FIG. 2 is another exemplary graph depicting relative ERCC1 expression in lung cancer patients with NSCLC in response to platinum-based therapy. The horizontal bar denotes ERCC1 median expression with quantitative values adjacent to the bar.

FIG. 3 is a graph of ERCC1 expression differences and statistical analysis across different disease states.

FIG. 4 is a graph depicting ERCC1 expression over time in lung cancer patients with NSCLC during platinum-based therapy.

FIG. 5 is a graph depicting dynamic changes in ERCC1 cfRNA in lung cancer patients with NSCLC during platinum-based therapy across different disease states.

FIG. 6 is a graph depicting PD-L1 expression over time in lung cancer patients with partial response (PR) to immune therapy with checkpoint inhibitors as indicated in the graph.

### DETAILED DESCRIPTION

There is an unmet need to evaluate tumor response by means other than a radiology test or biopsy from a residual tumor or new metastases. Advantageously, cfRNA (cell-free RNA that is derived from tumor cells and found as circulating RNA in biological fluids) can be extracted from plasma of cancer patients, and the inventors now discovered that measuring dynamic changes in gene expression of one or more specific genes, particularly in the context of expression of a reference gene (e.g., β-actin as a proxy for total cfRNA in the patient) can be used not only for disease detection, but also for evaluating disease status and/or predicting outcome to anti-tumoral therapy in advance of imaging. Viewed form a different perspective, the inventors have discovered that cfRNA can be employed as a sensitive, selective, and quantitative marker for diagnosis, monitoring of treatment, and even as discovery tool that allows repeated and non-invasive sampling of a patient. In addition, it should also be noted that contemplated systems and methods integrate with other omics analysis platforms, and especially GPS Cancer (that provides whole genome or exome sequencing, RNA sequence and expression analysis, and quantitative protein analysis) to establish a powerful primary analysis/monitoring combination tool in which alterations identified by an omics platform are non-invasively, molecularly monitored by systems and methods presented herein.

For example, and as is described in more detail below, the inventors discovered that levels of ERCC1 cfRNA (typically relative to expression levels of cfRNA for a housekeeping or other reference gene(s)) can be used to predict resistance or lack of response to treatment with platinum-based drugs (e.g., carboplatin). More specifically, the inventors discovered that a threshold difference of 4.2 between ERCC1 expression and beta-actin could be used as a cutoff for a prediction or assessment whether or not treatment with a platinum-based drug is likely successful (e.g., stable disease (SD), partial (PR) or complete response (CR)). In addition, it should be recognized that numerous expression levels other than ERCC1 are also deemed suitable, alone or in combination with ERCC1 cfRNA measurements. Therefore, it should be appreciated that one or more desired nucleic acids may be selected for quantitative cfRNA analysis in detection of cancer, ascertaining the disease stage, identifying specific mutations that may be cancer associated or patient specific. Alternatively, where discovery or scanning for new mutations or changes in expression of a particular gene is desired, real time quantitative PCR may be replaced by RNAseq to so cover at least a portion of the patient's transcriptome. Moreover, it should be appreciated that analysis can be performed static or over a time course with repeated sampling to obtain a dynamic picture without the need for biopsy of the tumor or a metastasis.

It should be noted that the term cfRNA includes full length RNA as well as fragments of full length RNA (which may have a length of 50-150 bases, 15-500 bases, or 500-1,000 bases, or more). Thus, cfRNA may represent a portion of an RNA, which may be between 100-80% of the full length RNA (typically mRNA), or between 80-60%, or between 60-40%, or between 40-20%, or even less. Moreover, it should be appreciated that the term cfRNA typically refers to a tumor-derived RNA (as opposed to an RNA from a non-tumor cell) and that the cfRNA may therefore be from a tumor cell of a solid tumor, a blood borne cancer, circulating tumor cells, and exosomes. Most typically, however, the cfRNA will be not be enclosed by a membrane (and as such be from a circulating tumor cell or exosome). Moreover, it should be appreciated that the cfRNA may be uniquely expressed in a tumor (e.g., as a function of drug resistance or in response to a treatment regimen, as a splice variant, etc.) or as a mutated form of a gene (e.g., as a fusion transcript, as a transcript of a gene having a single or multi-base mutation, etc.). Therefore, and viewed from a different perspective, contemplated cfRNA especially include transcripts that are unique to a tumor cell relative to a corresponding non-tumor cell, or significantly over- or under-expressed (e.g., at least 3-fold, or at least 5-fold, or at least 10-fold) in a tumor cell relative to a corresponding non-tumor cell, or have a mutation (e.g., missense or nonsense mutation leading to a neoepitope) relative to a corresponding non-tumor cell.

Most typically, suitable tissue sources include whole blood, which is preferably provided as plasma or serum. Thus, in a preferred embodiment, the ctDNA and/or ctRNA is isolated from a whole blood sample that is processed under conditions that preserve cellular integrity and stability of ctRNA as is further discussed below. Alternatively, it should be noted that various other bodily fluids are also deemed appropriate so long as ctDNA and/or ctRNA is present in such fluids. Appropriate fluids include saliva, ascites fluid, spinal fluid, urine, or any other types of bodily fluid, which may be fresh, chemically preserved, refrigerated or frozen.

The bodily fluid of the patient can be obtained at any desired time point(s) depending on the purpose of the cfRNA analysis. For example, the bodily fluid of the patient can be obtained before and/or after the patient is confirmed to have a tumor and/or periodically thereafter (e.g., every week, every month, etc.) in order to associate the ctDNA and/or ctRNA data with the prognosis of the cancer. In some embodiments, the bodily fluid of the patient can be obtained from a patient before, during, and/or after the cancer treatment (e.g., chemotherapy, radiotherapy, drug treatment, cancer immunotherapy, etc.). While it may vary depending on the type of treatments and/or the type of cancer, the bodily fluid of the patient can be obtained at least 24 hours, at least 3 days, at least 7 days after the cancer treatment. For more accurate comparison, the bodily fluid from the patient before the cancer treatment can be obtained less than 1 hour, less than 6 hours before, less than 24 hours before, less than a week before the beginning of the cancer treatment. In addition, a plurality of samples of the bodily fluid of the patient can be obtained during a period before and/or after the cancer treatment (e.g., once a day after 24 hours for 7 days, etc.). Therefore, where multiple samples are taken over the course of a predetermined interval (e.g., every day, every week, every two weeks, every month, etc.), dynamic changes can be assessed and trends identified that are indicative of disease state or predicted treatment response.

For example, for the analyses presented herein, specimens were accepted as 10 ml of whole blood drawn into cell-free RNA BCT® tubes or cell-free DNA BCT® tubes containing RNA or DNA stabilizers, respectively. Advantageously, cfRNA is stable in whole blood in the cell-free RNA BCT tubes for seven days while ctDNA is stable in whole blood in the cell-free DNA BCT Tubes for fourteen days, allowing time for shipping of patient samples from world-wide locations without the degradation of cfRNA or cfDNA.

Moreover, it is generally preferred that the cfRNA is isolated using RNA stabilization agents that will not or substantially not (e.g., equal or less than 1%, or equal or less than 0.1%, or equal or less than 0.01%, or equal or less than 0.001%) lyse blood cells. Viewed from a different perspective, the RNA stabilization reagents will not lead to a substantial increase (e.g., increase in total RNA no more than 10%, or no more than 5%, or no more than 2%, or no more than 1%) in RNA quantities in serum or plasma after the reagents are combined with blood. Likewise, these reagents will also preserve physical integrity of the cells in the blood to reduce or even eliminate release of cellular RNA found in blood cell. Such preservation may be in form of collected blood that may or may not have been separated. In less preferred aspects, contemplated reagents will stabilize cfDNA and/or cfRNA in a collected tissue other than blood for at 2 days, more preferably at least 5 days, and most preferably at least 7 days. Of course, it should be recognized that numerous other collection modalities are also deemed appropriate, and that the cfRNA and/or cfDNA can be at least partially purified or adsorbed to a solid phase to so increase stability prior to further processing.

It is generally preferred that the cfRNA is isolated using RNA stabilization reagents. While any suitable RNA stabilization agents are contemplated, preferred RNA stabilization reagents include one or more of a nuclease inhibitor, a preservative agent, a metabolic inhibitor, and/or a chelator. For example, contemplated nuclease inhibitors may include RNAase inhibitors such as diethyl pyrocarbonate, ethanol, aurintricarboxylic acid (ATA), formamide, vanadyl-ribonucleoside complexes, macaloid, heparin, bentonite, ammonium sulfate, dithiothreitol (DTT), beta-mercaptoethanol, dithioerythritol, tris(2-carboxyethyl)phosphene hydrochloride, most typically in an amount of between 0.5 to 2.5 wt %. Preservative agents may include diazolidinyl urea (DU), imidazolidinyl urea, dimethoylol-5,5-dimethylhydantoin, dimethylol urea, 2-bromo-2-nitropropane-1,3-diol, oxazolidines, sodium hydroxymethyl glycinate, 5-hydroxymethoxymethyl-1-laza-3,7-dioxabicyclo[3.3.0]octane, 5-hydroxymethyl-1-laza-3,7dioxabicyclo[3.3.0]octane, 5-hydroxypoly[methyleneoxy]methyl-1-1-aza-3,7-dioxabicyclo [3.3.0]octane, quaternary adamantine or any combination thereof. In most examples, the preservative agent will be present in an amount of about 5-30 wt %. Moreover, it is generally contemplated that the preservative agents are free of chaotropic agents and/or detergents to reduce or avoid lysis of cells in contact with the preservative agents.

Suitable metabolic inhibitors may include glyceraldehyde, dihydroxyacetone phosphate, glyceraldehyde 3-phosphate, 1,3-bisphosphoglycerate, 3-phosphoglycerate, phosphoenolpyruvate, pyruvate, and glycerate dihydroxyacetate, and sodium fluoride, which concentration is typically in the range of between 0.1-10 wt %. Preferred chelators may include chelators of divalent cations, for example, ethylenediaminetetraacetic acid (EDTA) and/or ethylene glycol-bis(3-aminoethyl ether)-N,N,N′,N′-tetraacetic acid (EGTA), which concentration is typically in the range of between 1-15 wt %.

Additionally, RNA stabilizing reagent may further include protease inhibitors, phosphatase inhibitors and/or polyamines. Therefore, exemplary compositions for collecting and stabilizing ctRNA in whole blood may include aurintricarboxylic acid, diazolidinyl urea, glyceraldehyde/sodium fluoride, and/or EDTA. Further compositions and methods for ctRNA isolation are described in U.S. Pat. Nos. 8,304,187 and 8,586,306, which are incorporated by reference herein.

Most preferably, such contemplated RNA stabilization agents for ctRNA stabilization are disposed within a test tube that is suitable for blood collection, storage, transport, and/or centrifugation. Therefore, in most typical aspects, the collection tube is configured as an evacuated blood collection tube that also includes one or more serum separator substance to assist in separation of whole blood into a cell containing and a substantially cell free phase (no more than 1% of all cells present). In general, it is preferred that the RNA stabilization agents do not or substantially do not (e.g., equal or less than 1%, or equal or less than 0.1%, or equal or less than 0.01%, or equal or less than 0.001%, etc.) lyse blood cells. Viewed from a different perspective, RNA stabilization reagents will not lead to a substantial increase (e.g., increase in total RNA no more than 10%, or no more than 5%, or no more than 2%, or no more than 1%) in RNA quantities in serum or plasma after the reagents are combined with blood. Likewise, these reagents will also preserve physical integrity of the cells in the blood to reduce or even eliminate release of cellular RNA found in blood cell. Such preservation may be in form of collected blood that may or may not have been separated. In some aspects, contemplated reagents will stabilize ctRNA in a collected tissue other than blood for at 2 days, more preferably at least 5 days, and most preferably at least 7 days. Of course, it should be recognized that numerous other collection modalities other than collection tube (e.g., a test plate, a chip, a collection paper, a cartridge, etc.) are also deemed appropriate, and that the cfRNA can be at least partially purified or adsorbed to a solid phase to so increase stability prior to further processing.

As will be readily appreciated, fractionation of plasma and extraction of cfRNA can be done in numerous manners. In one exemplary preferred aspect, whole blood in 10 mL tubes is centrifuged to fractionate plasma at 1600 rcf for 20 minutes. The so obtained clarified plasma fraction is then separated and centrifuged at 16,000 rcf for 10 minutes to remove cell debris. Of course, various alternative centrifugal protocols are also deemed suitable so long as the centrifugation will not lead to substantial cell lysis (e.g., lysis of no more than 1%, or no more than 0.1%, or no more than 0.01%, or no more than 0.001% of all cells). cfRNA is typically extracted from 2 mL of plasma using commercially available Qiagen reagents. For example, where cfRNA was isolated, the inventors used a second container that included a DNase that was retained in a filter material. Notably, the cfRNA also included miRNA (and other regulatory RNA such as shRNA, siRNA, and intronic RNA). Therefore, it should be appreciated that contemplated compositions and methods are also suitable for analysis of miRNA and other RNAs from whole blood. All nucleic acids are preferably kept in bar-coded matrix storage tubes, with RNA stored at −80° C. or reverse-transcribed to cDNA that is then stored at −4° C. Notably, so isolated ctRNA can be frozen prior to further processing.

With respect to quantitating/determining transcription strength (expression level) of the cfRNA it should be noted that such analysis can be performed in numerous manners. However, contemplated methods include quantification by digital PCR methods, absolute quantification methods using external standards, and most typically relative quantification methods using internal standards (e.g., expressed as 2^{ΔΔCt}). For example, real-time qPCR amplification can be performed using an assay in a 10 μL reaction mix containing 2 μL cDNA, primers, and probe. β-actin can be used as an internal standard for the input level of ct-cDNA. A standard curve of samples with known concentrations of each analyte can be included in each PCR plate as well as positive and negative controls for each gene. Delta Ct (dCT) can be calculated from the Ct value derived from quantitative PCR (qPCR) amplification for each analyte subtracted by the Ct value of β-actin for each individual patient's blood sample. Relative expression of patient specimens can then be calculated using a standard curve of delta Cts of serial dilutions of Universal Human Reference RNA set at a gene expression value of 10 (when the delta CTs were plotted against the log concentration of each analyte).

It should be particularly appreciated that such cfRNA centric systems and methods allow monitoring changes in markers and even drivers of a disease and/or to identify changes in markers or drug targets that may be associated with emerging resistance to chemotherapies as is shown in more detail below. For example, cfRNA presence and/or quantity of one or more specific gene (e.g., ERCC1 or PD-L1) may be used as a diagnostic tool to assess whether or not a patient may be sensitive to one or more platinum-based drug or checkpoint inhibitors.

Therefore, the inventors also contemplate that once a tumor is identified or detected, the prognosis of the tumor can be monitored by monitoring the types and/or quantity of cfRNAs in various time points. Once identified, cfRNAs, at least one of which is indicative of the disease, disease state, or treatability with a particular drug are isolated from a bodily fluid of the patient (typically whole blood, plasma, serum), and the quantity (and even subtype) of cfRNAs determined. As shown below, the inventors discovered that the quantity of cfRNA detected from the patient's bodily fluid can be a strong indicator of the disease, disease state, and treatability of the tumor. For example, increased quantities of ERCC1 and/or PD-L1 in a patient with lung cancer are indicative of resistance to treatment with a platinum based drug and indicative or likely treatment success with a checkpoint inhibitor.

Thus, it should be appreciated that the results from cfRNA quantification can not only be used as an indicator for the presence or absence of a specific cell or population of cells that gave rise to the measured cfRNA, but can also serve as an additional indicator of the state (e.g., genetic, metabolic, related to cell division, necrosis, and/or apoptosis) of such cells or population of cells. Indeed, where the results from other omics data and cfRNA quantification are employed as input data in pathway analysis and/or machine learning models, further insights with respect to suitable treatment options may be discovered. For example, suitable models include those that predict pathway activity (or activity of components of a pathway) in a single or multiple pathways. Thus, quantified cfRNA may also be employed as input data into models and modeling systems in addition to or as replacement for RNA data from transcriptomic analysis (e.g., obtained via RNAseq or cDNA or RNA arrays).

Particularly where the cfRNA is quantified over time, it is generally preferred that more than one measurement of the same (and in some cases newly identified) cfRNA are performed. For example, multiple measurements over time may be useful in monitoring treatment effect that targets the specific marker gene. Thus, such measurements can be performed before/during and/or after treatment. Among various other advantages, it should be appreciated that use of contemplated systems and methods simplifies treatment monitoring and even long term follow-up of a patient as target sequences are already pre-identified and target cfRNA can be readily surveyed using simple blood tests without the need for a biopsy. Such is particularly advantageous where micro-metastases are present or where the tumor or metastasis is at a location that precludes biopsy.

Further considerations, suitable cfRNAs, and methods are described in our copending International patent application with the serial numbers PCT/US18/22747, PCT/US18/30472, and PCT/US18/31764.

### Examples

Isolation of cfRNA from whole blood: Whole blood was obtained by venipuncture and 10 ml were collected into cell-free RNA BCT® tubes or cell-free DNA BCT® tubes (Streck Inc., 7002 S. 109^{th} St., La Vista Nebr. 68128) containing RNA or DNA stabilizers, respectively. The sample tubes were then centrifuged at 1,600 rcf for 20 minutes, plasma was withdrawn and further centrifuged at 16,000 rcf for 10 minutes to remove cell debris. Plasma was used to isolate cfRNA using commercially available RNA isolation kits following the manufacturer's protocol with slight modification. Specifically, DNA was removed from the sample in an on-column DNAse digest.

In an alternative approach, cfRNA was also obtained in an automated manner using a robotic extraction method on QiaSymphony instrumentation (Qiagen, 19300 Germantown Road; Germantown, Md. 20874), slightly modified to accommodate for DNA removal where desired. The robotic extraction maintained approximately 12% DNA contamination in the cfRNA sample. The inventors measured the relative expression of Excision Repair Cross-Complementing enzyme (ERCC1) versus beta actin in the same twenty-one NSCLC samples to determine whether there was a significant difference between the two extraction procedures. Notably, there was no statistical difference in the relative expression generated by the automated process and the manual process. Custom kit from Qiagen (QiaSymphony Circulating NA kit #1074536) included two virus extraction kits in one custom kit (the virus kits are called QiaSymphony DSP Virus/Pathogen Midi Kit Version 1 #937055). Analyses were run within single, proprietary program on Qiagen instrument (custom program protocol CF 2000S_CR21040_ID993; from Qiagen).

Quantification of cfRNA: Unless otherwise noted, quantification was performed using relative quantification via rtPCT and gene specific primer pairs along with primer pairs for beta-actin as internal control. For example, amplifications were performed using an assay in a 10 μL reaction mix containing 2 μL cDNA, primers, and probe. 3-actin can be used as an internal standard for the input level of ct-cDNA. A standard curve of samples with known concentrations of each analyte wad included in each PCR plate as well as positive and negative controls for each gene. Test samples were identified by scanning the 2D barcode on the matrix tubes containing the nucleic acids. Delta Ct (dCT) were calculated from the Ct value derived from quantitative PCR (qPCR) amplification for each analyte subtracted by the Ct value of 3-actin for each individual patient's blood sample. Relative expression of patient specimens was calculated using a standard curve of delta Cts of serial dilutions of Universal Human Reference RNA set at a gene expression value of 10 (when the delta CTs were plotted against the log concentration of each analyte). ctDNA was analyzed in a similar fashion.

Delta Cts vs. log₁₀ Relative Gene Expression (standard curves) for each gene test were captured over hundreds of PCR plates of reactions (historical reactions). A linear regression analysis was performed for each assay and used to calculate gene expression from a single point from the original standard curve going forward.

Assay Parameters—Accuracy: Accuracy of an exemplary PD-L1 Expression Assay was determined by comparing the results generated by the present PD-L1 assay (“LiquidGeneDx”) from 61 clinical samples against a digital PCR PD-L1 assay (lab developed reference method, an alternative PD-L1 detection assay). The results were used to determine the clinical sensitivity and clinical specificity of the assay. The accuracy results from the present PD-L1 assay and the digital PCR PD-L1 assay are summarized in Table 1.

TABLE 1

| | Positive Agreement | Negative Agreement |
|---|---|---|
| | (LiquidGeneDx vs Digital PCR) | (LiquidGeneDx vs Digital PCR) |
| PD-L1 | 91% | 94% |

Assay Parameters—Limit of Detection (LOD): Analytical sensitivity of the present PD-L1 assay (“LiquidGeneDx”) was determined by 20 replicates at a 95% detection rate. cfRNA was extracted from patients' plasma, reverse-transcribed using random hexamers to cDNA and pre-amplified using Thermo Fisher's pre-amplification product Taqman® Preamp Master Mix with PD-L1 and beta-actin primers for 10 cycles per the manufacturer's instructions. The resulting pre-amplified cDNA was diluted in 2-fold increments with cDNA from patients' plasma negative for PD-L1. All dilution samples were examined by LiquidGeneDx for the minimum amount of PD-L1 cDNA required for amplification and successful PCR. Then 20 replicates at the presumptive LOD level were used to confirm the final LOD. The limit of detection (LOD) acceptance criteria in this study was determined as the lowest concentration at which all 20 replicates generated a 95% above the detection rate. If 20 replicates could not generate a 95% above detection rate, the next higher concentration of dilution samples were used as presumptive LOD to repeat with 20 replicates. A summary of LOD study results is shown in Table 2 in which the * denotes the final LOD.

TABLE 2

| PD-L1 Dilution | Valid Positive Results/Total Tested | | | | | |
|---|---|---|---|---|---|---|
| Sample | 1.884 ng | 0.941 ng | 0.471 ng | 0.236 ng | 0.118 ng* | 0.059 ng |
| PD-L1 | 4/4 | 4/4 | 4/4 | 4/4 | 20/20 | 15/20 |
| Expression | | | | | | |

Assay Parameters—Linear Range: Quantitative linear range of the present PD-L1 assay (“LiquidGeneDx”) was determined by diluting PD-L1-positive patients' cDNA from cfRNA into a pooled negative matrix (PD-L1-negative cDNA from cfRNA). ct RNA was extracted from patients' plasma, reverse-transcribed using random hexamers to cDNA and pre-amplified using Thermo Fisher's pre-amplification product Taqman® Preamp Master Mix with PD-L1 and beta-actin primers for 10 cycles per the manufacturer's instructions. The resulting pre-amplified cDNA was diluted in 2-fold increments with cDNA from patients' plasma negative for PD-L1. All dilution samples were examined by LiquidGeneDx PD-L1 to determine its quantitative linear range. The linear portion of the line extended to a Ct of approximately 32.5. Beta-actin and PD-L1 slopes were concordant.

Patients: Blood was drawn from patients at approximately 6-week intervals under various therapies, with CT scans at 3-month intervals. Total cfRNA was extracted from patient plasma and reverse transcribed to cDNA. Levels of β-actin, ERCC1 and PD-L1 were quantitated across multiple blood draws by RT-qPCR and correlated with patient response (PR/SD/PD), as determined by CT scans.

Results: A total of 24 NSCLC patients were enrolled in a 1-year clinical study. Non-SCC comprised 87% (21/24). 19 patients completed the first two cycles of therapy. 1 patient with PR had decreasing levels of cfRNA, 10 patients achieved SD with decreasing or no change while 6/8 patients with PD had increasing levels of cfRNA. CfRNA levels were predictive of disease status about 4 weeks in advance of imaging in 6/19 patients and matched with disease status in 8/19 patients (74% concordance). Dynamic changes in PD-L1 expression correlated with response to nivolumab in 3/4 patients. In 2/4 patients with SD, PD-L1 remained undetected after therapy, whereas 1 patient continued to have PD despite loss of PD-L1. PD-L1 was undetectable in a patient initially with PD on nivolumab who achieved SD after one cycle of nivolumab plus radiation. Changing ERCC1 expression correlated with platinum-based therapy outcome in 8/8 patients. 4/4 patients with PD on pemetrexed/carboplatin had an increase in ERCC1. 4/4 patients with lower or decreasing levels of ERCC1 achieved PR or SD. In the only patient achieving PR, ERCC1 became undetectable during treatment.

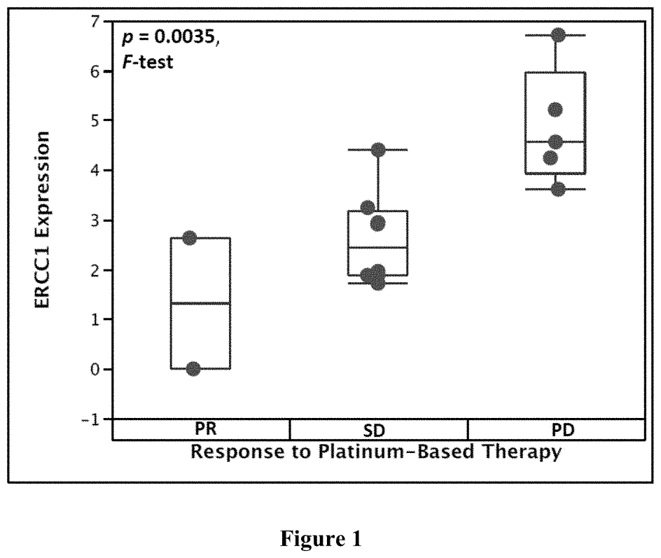

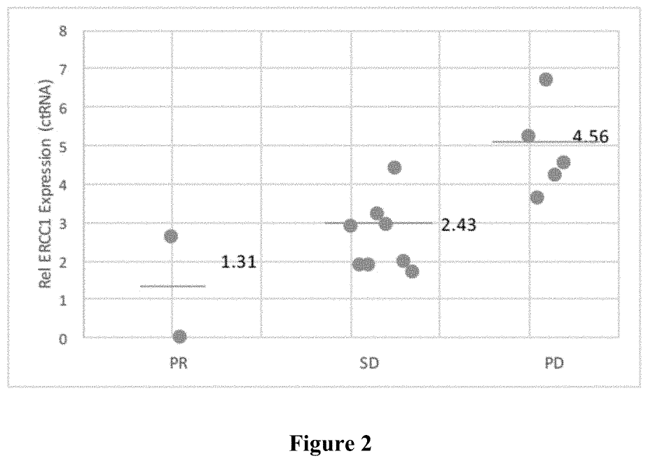

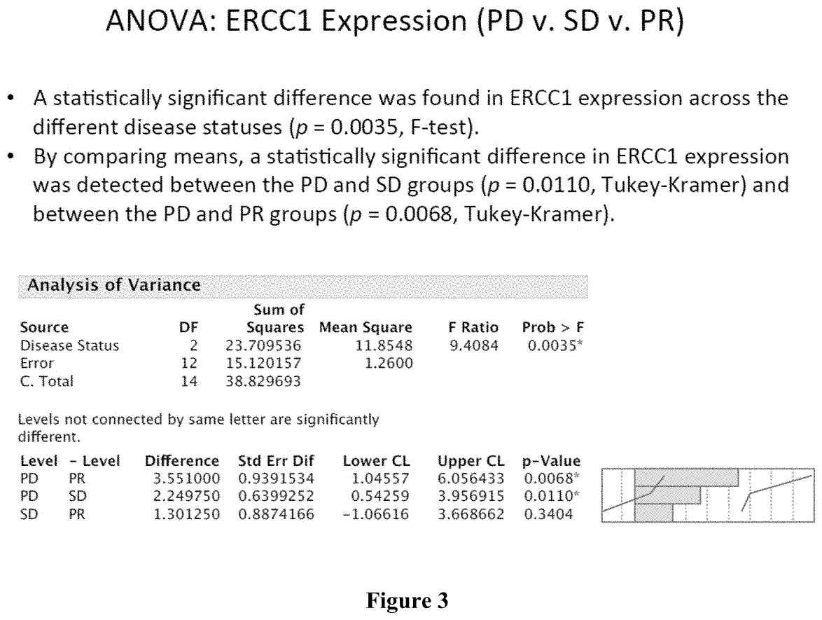

FIG. 1 is a graph depicting patient treatment response to a platinum-based treatment (here carboplatin treatment) by relative ERCC1 cfRNA expression. As can be readily seen from the graph, the partial response groups and the stable disease groups were statistically significantly distinguishable from one another as well as from disease progression. FIG. 2 is a graph depicting raw data and median expression as a function of PR, SD, and PD, while FIG. 3 provides further data and analysis. Of course, it should also be appreciated that detection and/or quantification of the above indicators need not be limited to fRNA detection/quantification, but that analysis may also include a pathway analysis where pathways that include ERCC1 and/or PD-1 signaling are examined for (de)activation. Likewise, additional (or alternative) analyses may include protein analyses, and especially quantitative protein analyses such as mass spectroscopic analyses (e.g., selective reaction monitoring and variations thereof).

In further studies, blood was drawn from patients at approximately 6-week intervals under various therapies (platinum-based therapy and immune therapy with checkpoint inhibitors), with CT scans at 3-month intervals. Total cfRNA was extracted from patient plasma and reverse transcribed to cDNA. Levels of β-actin, ERCC1 and PD-L1 were quantitated across multiple blood draws by RT-qPCR and correlated with patient response (PR/SD/PD), as determined by CT scans.

A total of 29 NSCLC patients were enrolled in a 1-year clinical study. Non-SCC comprised 86% (25/29). 23 patients completed the first two cycles of therapy. 2/3 patients with PR had decreasing levels of cfRNA, 10 patients achieved SD with decreasing or no change while 7/9 patients with PD had increasing levels of cfRNA. CfRNA levels were predictive of disease status about 4 weeks in advance of imaging in 7/23 patients and matched disease status in another 10/23 patients (74% total concordance). Dynamic changes in PD-L1 expression correlated with response to immunotherapy in 6/7 patients. In 2 patients with SD, PD-L1 remained undetected after therapy, whereas 1 patient showed PD despite loss of PD-L1. PD-L1 increased in another patient with PD and decreased in 2 patients with PR. Changing ERCC1 expression correlated with platinum-based therapy outcome in 9/10 patients. 4/5 PD patients on carboplatin/pemetrexed had increases in ERCC1; 5/5 patients with lower/decreasing ERCC1 achieved PR or SD.

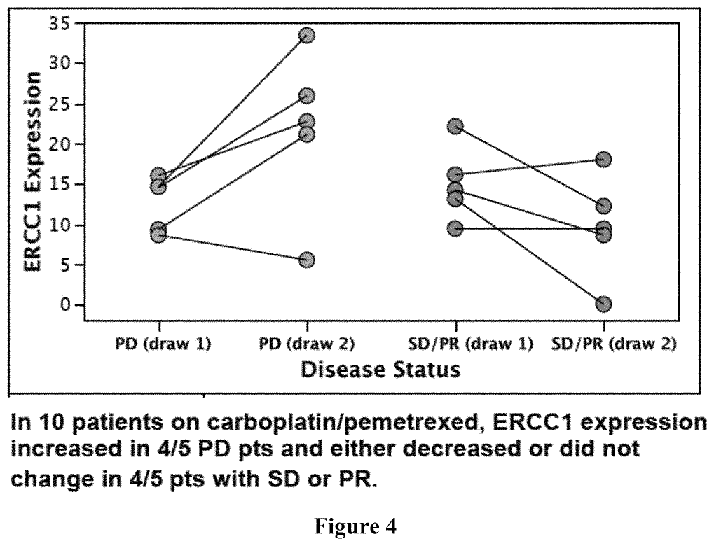

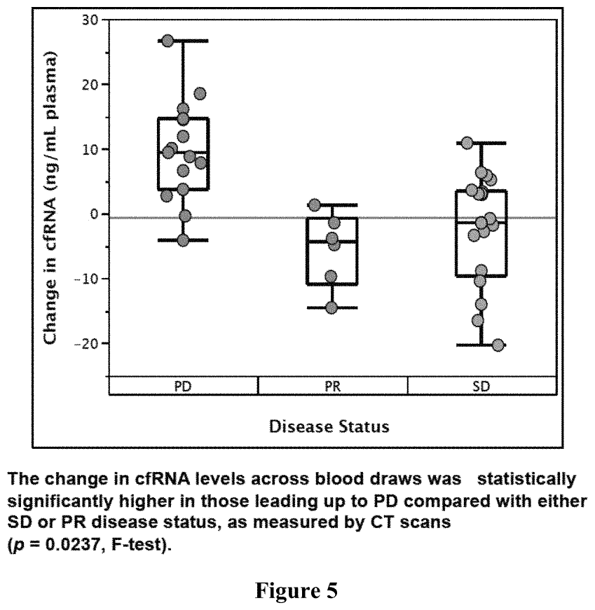

As can be readily seen from the Figures, quantities of ERCC1 cfRNA dynamically changed over time with a significant increase of ERCC1 cfRNA in substantially all patients experiencing progressive disease. Conversely, ERCC1 cfRNA significantly decreased in substantially all patients experiencing stable disease or at least partial response as shown in FIG. 4. A statistical analysis of the change is shown in the plot of FIG. 5 where it is readily apparent that dynamic changes in ERCC1 cfRNA correlates with the disease status as indicated. Notably, a positive change in ERCC1 cfRNA was associated with progressive disease, while a negative change in ERCC1 cfRNA was associated with partial response. Where the disease status was stable disease, no significant change was overall observed.

For example, where the disease state is partial response, the relative expression of ERCC1 cfRNA (typically against beta-actin) is at or below 2.0, or at or below 1.8, or at or below 1.5, or at or below 1.3. Where the disease state is stable disease, the relative expression of ERCC1 cfRNA (typically against beta-actin) is between 1.5 and 2.0, or between 2.0 and 2.5, or between 1.8 and 2.8, or between 2.0 and 2.8, or between 2.2 and 2.6, or between 2.2 and 3.0, or between 2.5 and 3.5. Viewed from a different perspective, the relative expression of ERCC1 cfRNA (typically against beta-actin) is typically more than 1.8, or more than 2.0, or more than 2.4, or more than 2.6, or more than 2.8, but less than 4.0, or less than 3.8, or less than 3.6, or less than 3.3, or less than 3.0. On the other hand, where the disease state is progressive disease, the relative expression is typically at or above 3.2, or at or above 3.4, or at or above 3.8, or at or above 4.0, or at or above 4.3, or at or above 4.5, or at or above 4.7, or even higher.

Similarly, where the ERCC1 cfRNA levels are measured over time, a difference in expression level (ng/ml plasma) of at least +3, or at least +5, or at least +7, or at least +10, or at least +12 is indicative of progressive disease, while a difference in expression level (ng/ml plasma) of less than −1, or less than −2, or less than −3, or less than −5, or less than −10× is indicative of at least partial response. On the other hand, a difference in expression level (ng/ml plasma) of between −1 and +1, or between −3 and +3, or between −3 and +2, or between −5 and +1, or between −7 and 0, or between −10 and 0 is indicative of stable disease. Such changes are typically observed over at least 1 month, or at least three months, or at least six months, or at least 1 year. Viewed from a different perspective, such dynamic changes are typically observed between start and conclusion of treatment.

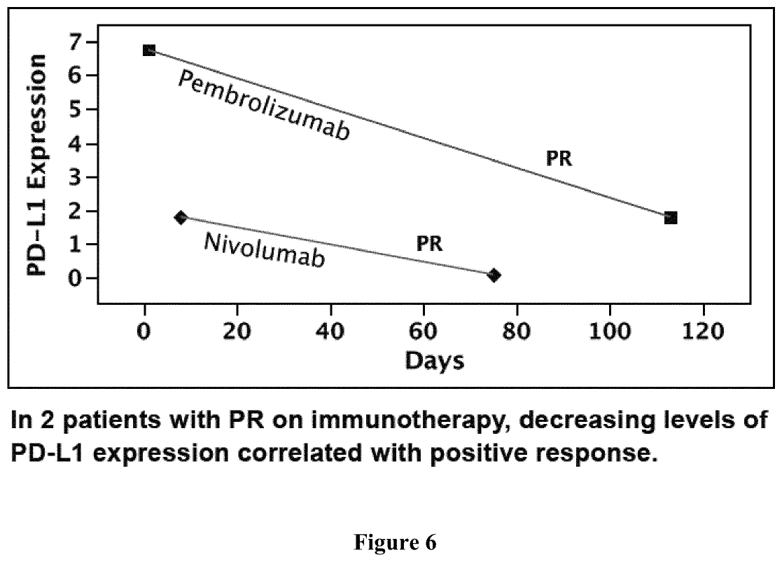

When these patients were also evaluated for PD-L1 cfRNA expression levels, it was observed that relative expression levels for PD-L1 cfRNA correlated with positive treatment responses as is shown for two patients in FIG. 6. Here, a decrease of PD-L1 cfRNA was strongly associated with partial response.

Therefore, it should be recognized that disease status for a cancer, and especially lung cancer, can be ascertained before, during and after treatment with a platinum-based drug (e.g. carboplatin, cisplatin, etc.) by quantification of relative ERCC1 cfRNA expression as well as by dynamic changes in ERCC1 cfRNA expression. Similar results were obtained for PD-L1 cfRNA. Thus, it should be noted that ERCC1 and PD-L1 expression in cfRNA can be used to monitor response to platinum based and immuno-therapy.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. A method of monitoring or predicting clinical response with respect to a disease state of a cancer in a patient subject to treatment with a platinum-based drug, comprising:
quantitating relative expression of ERCC1 cfRNA in a bodily fluid of the patient diagnosed with the cancer.

2. The method of claim 1 wherein the cancer is lung cancer.

3. The method of claim 1, wherein the relative expression of ERCC1 cfRNA is quantified relative to beta-actin RNA or Universal Human Reference RNA.

4. The method of claim 1, further comprising a step of generating an indication when the relative expression of ERCC1 cfRNA is above a level indicative for stable disease.

5. The method of claim 4, wherein the indication is predicted resistance or lack of response to the treatment with the platinum-based drug.

6. The method of claim 1, further comprising a step of generating an indication when the relative expression of ERCC1 cfRNA is at or below a level indicative for stable disease.

7. The method of claim 6, wherein the indication is predicted partial or full response to the treatment with the platinum-based drug.

8. The method of claim 1, further comprising a step of quantitating relative expression of PD-L1 cfRNA in the bodily fluid of the patient diagnosed with the cancer.

9. The method of claim 8, wherein the patient is further subject to treatment with a checkpoint inhibitor.

10. The method of claim 1, further comprising, at least one week after the step of quantitating relative expression of ERCC1 cfRNA a second step of quantitating the relative expression of ERCC1 cfRNA in the bodily fluid.

11. The method of claim 10, further comprising detecting a dynamic change of ERCC1 cfRNA using the steps of quantitating the relative expression of ERCC1 cfRNA in the bodily fluid.

12. The method of claim 1, the disease state is partial response when the relative expression is at or below 1.5.

13. The method of claim 1, wherein the disease state is stable disease when the relative expression is between 1.8 and 2.8.

14. The method of claim 1, wherein the disease state is progressive disease when the relative expression is at or above 3.8.

15-35. (canceled)
